(19) United States
(12) Patent Application Publication
(10) Pub. No.: US 2008/0213209 A1
(43) Pub. Date: Sep. 4, 2008

(54) Process for Producing Cross-Linked Material of Polylactic Acid and Cross-Linked Material of Polylactic Acid

(76) Inventors: Shinichi Kanazawa, Osaka (JP); Kiyoshi Kawano, Osaka (JP)

Correspondence Address: MCDERMOTT WILL & EMERY LLP, 600 13TH STREET, N.W., WASHINGTON, DC, 20005-3096, US

(21) Appl. No.: 11/886,430

(22) PCT Filed: Mar. 2, 2006
(86) PCT No.: PCT/JP2006/303957

§ 371 Date: Sep. 14, 2007

(30) Foreign Application Priority Data
Mar. 14, 2005 (JP) 2005-071725
Aug. 30, 2005 (JP) 2005-249361

(51) Int. Cl. A61K 31/74; B29C 71/04; C08J 3/28

(52) U.S. Cl. 424/78.31; 522/3; 264/405

(57) ABSTRACT

A cross-linked material of polylactic acid produced by preparing a polylactic acid composition by mixing polylactic acid with at least a plasticizer containing either a rosin derivative or a dicarboxylic acid derivative and/or a glycerin derivative and cross-linking monomer, and kneading the resulting mixture; preparing a polylactic acid molded product by molding the composition into a desired shape; and then cross-linking the molded product by irradiation of ionizing radiation.

### TECHNICAL FIELD

The present invention relates to a method of producing a biodegradable cross-linked material of polylactic acid, and a cross-linked material of polylactic acid produced thereby, and more particularly to a biodegradable cross-linked material of polylactic acid useful in fields in which plastic products including structures such as films, containers and chassis, and plastic components are used, in particular for resolving issues concerning disposal of used plastic products.

### BACKGROUND ART

Petro-synthesis polymeric materials used in a wide variety of films and containers are currently causing several social problems such as, in the disposal process thereof alone, global warming due to heat and gases exhausted in incineration processes, adverse effects on foods and human health brought about by toxic substances existing in combustion gases and combustion residues, and depletion of available waste burying sites.

Recently, biodegradable polymeric materials as represented by starch and polylactic acid have been attracting attention because of their applicability as materials for resolving such problems in the disposal process of petro-synthesis polymeric materials. Biodegradable polymeric materials generate less heat than petro-synthesis polymeric materials when incinerated and maintain natural degradation and resynthesis cycles, thus exerting no adverse effect on the global environment including ecologies. Compared to other kinds of biodegradable polymeric materials, aliphatic polyester resins have recently come to particular attention because of their performance in strength and processability comparable to that of petro-synthesis polymeric materials. In particular, polylactic acid is produced of plant-derived starch unlike other kinds of aliphatic polyester resins and the recent mass-production thereof has significantly lowered its manufacturing cost to less than that of other kinds of biodegradable polymeric materials. As a result, applications of polylactic acid have been extensively investigated.

However, a film of polylactic acid is very stiff and has little flexibility and adhesive properties at temperatures lower than 60° C., its glass transition temperature, but is too flexible to maintain its shape at temperatures equal to or higher than 60° C., the glass transition temperature, thus being difficult to use in practice.

Though the temperature of air and water in nature do not often increase to 60° C., for example, the interior space and windows of closed automobiles may be heated to such a temperature in midsummer.

Therefore, the significant change in the characteristics, i.e., the fact that the material is stiff and fragile at temperatures equal to or lower than 60° C. but is too soft to maintain its shape at temperatures equal to or higher than 60° C., is a serious disadvantage.

This significant change in the characteristics is attributable to the crystalline structure of polylactic acid. More specifically, when cooled at a usual cooling rate after the melt-forming process, polylactic acid is negligibly crystallized and a large portion thereof becomes solidified in an amorphous state. The crystallized portions of polylactic acid, whose melting point is as high as 160° C., can not easily melt, whereas the amorphous portions accounting for the major portion of the entire product start to move without restriction at temperatures close to 60° C., its glass transition temperature. Thus the characteristics of polylactic acid greatly change at temperatures near 60° C., the glass transition temperature.

As an example of materials for making films and containers, which are required to be flexible at room temperature, Japanese Unexamined Patent Application Publication No. 2004-277682 (Patent Document 1) proposes a flexible material obtained by modification of polylactic acid which has high stiffness and little flexibility at temperatures lower than 60° C.

Patent Document 1 described above discloses a modified biodegradable resin obtained by mixing 100 parts by weight of a polylactic-acid-containing biodegradable resin with 3 to 80 parts by weight of a rosin compound.

The patent document states that the abovementioned modified biodegradable resin has improved flexibility and adhesive properties, but does not discuss whether the shape of the resin product can be maintained at temperatures equal to or higher than the glass transition temperature of polylactic acid, thus leaving room for improvement. Furthermore, the flexibility of the resin is also insufficient because the percentage elongation after fracture of the resin is in the range of approximately 1.7% to 3.4%, as seen in Table II on Page (7) of Patent Document 1.

Patent Document 1: Japanese Unexamined Patent Application Publication No. 2004-277682

### DISCLOSURE OF INVENTION

### Problems to be Solved by the Invention

An object of the present invention is to provide a cross-linked material of polylactic acid wherein the change in strength is small around 60° C., the glass transition temperature of polylactic acid; the flexibility comparable to that of general-purpose petro-synthesis polymeric materials is achieved at temperatures lower than 60° C., the glass transition temperature; and the shape of its product can be maintained even at high temperatures equal to or higher than 60° C. Another object of the present invention is to provide a method of producing the cross-linked material of polylactic acid described above.

### Means for Solving the Problems

To achieve the objects above, the first aspect of the present invention provides a method of producing a cross-linked material of polylactic acid comprising

a step of producing a polylactic acid composition by mixing polylactic acid, at least one plasticizer containing a rosin derivative, and cross-linking monomer, and then kneading the resulting mixture;

a step of producing a polylactic acid molded product by molding the polylactic acid composition obtained in the previous step into a desired shape; and

a step of cross-linking the polylactic acid molded product obtained in the previous step by irradiation of ionizing radiation.

The second aspect of the present invention provides a method of producing a cross-linked material of polylactic acid comprising

a step of producing a polylactic acid composition by mixing polylactic acid, a plasticizer containing a dicarboxylic acid derivative and/or a glycerin derivative, and cross-linking monomer, and then kneading the resulting mixture;

a step of producing a polylactic acid molded product by molding the polylactic acid composition obtained in the previous step into a desired shape; and

a step of cross-linking the polylactic acid molded product obtained in the previous step by irradiation of ionizing radiation.

The inventers conducted comprehensive investigations and found the following facts: to achieve a sufficient flexibility at temperatures lower than 60° C., the glass transition temperature of polylactic acid, the addition of a plasticizer containing the derivative is preferable; to maintain the shape of the product at temperatures equal to or higher than 60° C., cross-linking of polylactic acid chains is preferable; and preferable cross-linking means is irradiation of ionizing radiation.

Examples of a plasticizer for polylactic acid include a plasticizer that is a liquid at room temperature such as glycerin, ethylene glycol and triacetyl glycerin, as well as a plasticizer that is a solid at room temperature such as biodegradable resins including polyglycolic acid and polyvinyl alcohol. However, the polylactic acid chains in the present invention are cross-linked by irradiation of ionizing radiation, so that the plasticizer used is required not to interfere with the cross-linking reactions using ionizing radiation while being resistant to ionizing radiation.

From this point of view, the inventors examined several plasticizers and found that a plasticizer containing a rosin derivative as its main ingredient hardly interferes with the cross-linking reactions using ionizing radiation and has a resistance to ionizing radiation. As a result, the first aspect of the present invention was established.

Also, the inventers found that a plasticizer containing a dicarboxylic acid derivative and/or a glycerin derivative hardly interferes with the cross-linking reactions using ionizing radiation and even a small amount of the plasticizer can render flexibility to polylactic acid. As a result, the second aspect of the present invention was established.

In particular, the inventors found that, when mixed with polylactic acid and cross-linking monomer, molecules of a plasticizer containing a glycerin derivative is cross-linked to the polylactic acid chains during irradiation of ionizing radiation, thus preventing bleed, the biggest problem in using a plasticizer, from occurring.

In other words, in the method of producing a cross-linked material of polylactic acid according to the first aspect of the present invention established based on the findings above, a polylactic acid composition is produced by mixing polylactic acid with at least one plasticizer containing a rosin derivative and cross-linking monomer, and then kneading the resulting mixture; the obtained polylactic acid composition is formed into a desired shape; and then the polylactic acid chains are cross-linked to each other by irradiation of ionizing radiation, as described earlier.

Also, in the method of producing a cross-linked material of polylactic acid according to the second aspect of the present invention, a polylactic acid composition is produced by mixing polylactic acid with a plasticizer containing a dicarboxylic acid derivative and/or a glycerin derivative and cross-linking monomer, and then kneading the resulting mixture; the obtained polylactic acid composition is formed into a desired shape; and then polylactic acid is cross-linked by irradiation of ionizing radiation, as described earlier.

Examples of the rosin derivative used in the method of producing a cross-linked material of polylactic acid according to the first aspect of the present invention include raw material rosins such as a gum rosin, a wood rosin and a tall oil rosin; stabilized rosins and polymeric rosins obtained via disproportionation or hydrotreatment of the raw material rosins; as well as rosin esters, strengthened rosin esters, rosin phenols and rosin modified phenol resins. These rosin derivatives may be used separately or in combination of two or more kinds.

The rosin esters represent compounds formed as a result of an esterification reaction between raw material rosin and an alcohol. The rosin phenols represent compounds formed by addition of phenol to a raw material rosin and subsequent thermopolymerization and optional esterification. In addition, any compounds formed as a result of an addition reaction wherein an alkylene oxide such as ethylene oxide and propylene oxide is added to one of the raw material rosins may be used just like the rosin esters described above.

In addition, examples of alcohols used in the abovementioned esterification include known monohydric alcohols such as methanol;

known dihydric alcohols and monoalkyl ethers thereof such as trimethylolethane, trimethylolpropane, ethylene glycol, ethylene glycol monoalkyl ether, diethylene glycol, diethylene glycol monoalkyl ether, triethylene glycol, triethylene glycol monoalkyl ether, polyethylene glycol, polyethylene glycol monoalkyl ether, propylene glycol, propylene glycol monoalkyl ether, dipropylene glycol, dipropylene glycol monoalkyl ether, tripropylene glycol, tripropylene glycol monoalkyl ether, polypropylene glycol, and polypropylene glycol monoalkyl ether; and known polyhydric alcohols having three or more hydroxyl groups such as glycerin and pentaerythritol, which may be used separately or in combination of two or more kinds.

The plasticizer described earlier contains a rosin derivative as its main ingredient, but may further contain any ingredient other than the rosin derivative unless the ingredient has an adverse effect on achieving the objects of the present invention. The content ratio of the rosin derivative in the plasticizer is preferably 80 weight percent (wt %) or more, more preferably 90 wt % or more, and most preferably 100 wt %.

The rosin derivative-containing plasticizer is mixed with 100 wt % of polylactic acid preferably at a content ratio from 15 wt % to 30 wt %.

The reason why the content ratio of the abovementioned plasticizer is at least 15 wt % is the fact that content ratios of the plasticizer lower than 15 wt % can not improve the flexibility of polylactic acid sufficiently. On the other hand, the reason why the content ratio of the plasticizer is equal to or lower than 30 wt % is the concern that content ratios of the plasticizer higher than 30% may cause bleed, i.e., effluence of the plasticizer after the molding process, to occur.

Meanwhile, examples of the glycerin derivative used in the method of producing a cross-linked material of polylactic acid according to the second aspect of the present invention include triacetyl glyceride also known as triacetin, diacetyl monoester glyceride as represented by RIKEMAL PL from Riken Vitamin Co., Ltd., and diglycerol tetra acetate.

Also, examples of the abovementioned dicarboxylic acid derivative include oxalic acid, malonic acid, succinic acid, adipic acid, sebacic acid, glutaric acid, decanedicarboxylic acid, terephthalic acid and isophthalic acid. An example of commercial dicarboxylic acid derivatives is DAIFFATY-101 manufactured by Daihachi Chemical Industry Co., Ltd.

The plasticizer containing the dicarboxylic acid derivative and the other plasticizer containing the glycerin derivative may be used alone or in combination thereof, or further contain any other ingredient.

In any of these cases, the plasticizer used in the present invention contains the dicarboxylic acid derivative and/or the glycerin derivative as its main ingredient(s), and the content ratio thereof in 100 wt % of the entire plasticizer is preferably 80 wt % or more, more preferably 90 wt % or more, and most preferably 100 wt %.

Furthermore, the abovementioned plasticizer containing the dicarboxylic acid derivative and/or the glycerin derivative is mixed with 100 wt % of polylactic acid preferably at a content ratio from 3 wt % to 30 wt %.

The reason why the content ratio of the abovementioned plasticizer is at least 3 wt % is the fact that content ratios of the plasticizer lower than 3 wt % can not improve the flexibility of polylactic acid sufficiently. On the other hand, the reason why the content ratio of the plasticizer is equal to or lower than 30 wt % is the concern that content ratios of the plasticizer higher than 30% may cause bleed, i.e., effluence of the plasticizer after the molding process, to occur.

Examples of polylactic acid used in the first and second aspects of the present invention include polylactic acid consisting of L-lactic acid, polylactic acid consisting of D-lactic acid, polylactic acid obtained by polymerization of a mixture of L- and D-lactic acids, and combinations of two or more kinds thereof. It should be noted that monomers constituting polylactic acid, i.e., L- and D-lactic acids, may be chemically modified.

Polylactic acid preferably used in the present invention is homopolymer such as those described above, but lactic acid copolymer obtained by copolymerization between either lactic acid monomer or lactide and other components that can be copolymerized with lactic acid or lactide may also be used. Examples of the abovementioned “other components” used to form the copolymer include hydroxycarboxylic acids such as glycolic acid, 3-hydroxybutyric acid, 5-hydroxyvaleric acid and 6-hydroxycaproic acid; dicarboxylic acids such as succinic acid, adipic acid, sebacic acid, glutaric acid, decanedicarboxylic acid, terephthalic acid and isophthalic acid; polyhydric alcohols such as ethylene glycol, propanediol, octanediol, dodecanediol, glycerin, sorbitan and polyethylene glycol; and lactones such as glycolide, ε-caprolactone and δ-butyrolactone.

In addition, the kind of the cross-linking monomer mixed with polylactic acid in the first and second aspects of the present invention is not particularly limited as long as it can serve as a cross-linker in response to irradiation of ionizing radiation. For example, acrylic-, methacrylic- or allylic-type cross-linking monomer may be used.

Examples of the acrylic- or methacrylic-type cross-linking monomer include 1,6-hexanediol di(meth)acrylate, 1,4-butanediol di(meth)acrylate, trimethylolpropane tri(meth)acrylate, ethylene oxide modified trimethylolpropane tri(meth)acrylate, propylene oxide modified trimethylolpropane tri(meth)acrylate, ethylene oxide modified bisphenol A di(meth)acrylate, diethylene glycol di(meth)acrylate, dipentaerythritol hexaacrylate, dipentaerythritol monohydroxy pentaacrylate, caprolactone modified dipentaerythritol hexaacrylate, pentaerythritol tri(meth)acrylate, pentaerythritol tetra(meth)acrylate, polyethyleneglycol di(meth)acrylate, tris(acryloxyethyl)isocyanurate and tris(methacryloxyethyl)isocyanurate.

Examples of the allylic-type cross-linking monomer include triallylisocyanurate, trimethallylisocyanurate, triallylcyanurate, trimethallylcyanurate, diallylamine, triallylamine, diacrylchlorentate, allyl acetate, allyl benzoate, allyl dipropyl isocyanurate, allyl octyl oxalate, allyl propyl phthalate, bityl allyl maleate, diallyl adipate, diallyl carbonate, diallyl dimethyl ammonium chloride, diallyl fumarate, diallyl isophthalate, diallyl malonate, diallyl oxalate, diallyl phthalate, diallyl propyl isocyanurate, diallyl sebacate, diallyl succinate, diallyl terephthalate, diallyl tartrate, dimethyl allyl phthalate, ethyl allyl maleate, methyl allyl fumarate, methyl methallyl maleate and diallyl monoglycidyl isocyanurate.

Cross-linking monomer preferably used in the first and second aspects of the present invention is the allylic-type cross-linking monomer, which exerts an excellent cross-linking performance even when used at a relatively low concentration. In particular, triallylisocyanurate (hereinafter, TAIC) displays an excellent cross-linking performance for polylactic acid and also has cross-linking properties for polybutylene adipate terephthalate (PBAT), thus being particularly preferably used. In addition, triallylcyanurate, which can be easily transformed into and reproduced from TAIC by heating, provides the substantially same effect as TAIC.

In the first aspect of the present invention, the abovementioned cross-linking monomer is mixed with 100 wt % of polylactic acid preferably at a content ratio from 3 wt % to 8 wt %.

A content ratio of the cross-linking monomer as low as 0.5 wt % can exert a cross-linking effect. However, to consistently achieve one of the objects of the present invention, i.e., the effect of maintaining the product strength at high temperatures equal to or higher than 60° C., the content ratio equal to or higher than 3 wt % is preferable. On the other hand, the reason why the content ratio of the cross-linking monomer is equal to or lower than 8 wt % is the fact that content ratios of the cross-linking monomer higher than 8 wt % make it difficult to mix the full amount of the cross-linking polymer and polylactic acid uniformly, thus offering only a slight advantage in the effect.

In addition, to increase the content ratio of polylactic acid so as to improve the biodegradability, the content ratio of the cross-linking monomer is preferably 5 wt % or less, and thus the most preferable content ratio of the cross-linking monomer is in the range of 3 wt % to 5 wt %.

On the other hand, the second aspect of the present invention preferably contains the abovementioned cross-linking monomer at a content ratio from 3 to 15 parts by weight in 100 parts by weight of polylactic acid.

The reason why the content ratio of the cross-linking monomer is at least 3 parts by weight is the concern that content ratios of the cross-linking monomer lower than 3 parts by weight can not exert cross-linking performance sufficiently, thus resulting in a reduced strength of the biodegradable cross-linked product at high temperatures equal to or higher than 60° C., or in the worst case, the shape of the product can not be maintained. On the other hand, the reason why the content ratio of the cross-linking monomer is equal to or less than 15 parts by weight is the fact that content ratios of the cross-linking monomer higher than 15 parts by weight offer only a slight advantage in the cross-linking performance.

As for the first aspect of the present invention, the polylactic acid composition produced in the abovementioned first step may contain additional ingredients other than the polylactic acid, the plasticizer containing a rosin derivative as its main ingredient, and the cross-linking monomer, unless the ingredient has an adverse effect on achieving the objects of the present invention. Meanwhile, as for the second aspect of the present invention, the polylactic acid composition produced in the first step may contain additional ingredients other than the polylactic acid, the plasticizer containing a dicarboxylic acid derivative and/or a glycerin derivative, and the cross-linking monomer, unless the ingredient has an adverse effect on achieving the objects of the present invention.

For example, any biodegradable resin other than polylactic acid may be added in the composition. Examples of the biodegradable resin other than polylactic acid include synthetic polymers such as a lactone resin, aliphatic polyesters and polyvinyl alcohol, and natural polymers such as natural linear polyester resins, e.g., polyhydroxy butyrate/valerate.

Also, biodegradable synthetic polymer and/or natural polymer may be mixed with the composition as far as the addition of the polymer does not impair the fusing characteristics. Examples of the biodegradable synthetic polymer include cellulose esters such as cellulose acetate, cellulose butyrate, cellulose propionate, cellulose nitrate, cellulose sulfate, cellulose acetate butyrate and cellulose acetate nitrate; and polypeptides such as polyglutamic acid, polyaspartic acid and polyleucine. Examples of the natural polymer include starch, e.g., raw starch such as maize starch, wheat starch and rice starch, and processed starch such as acetic ester starch, methyl ether starch and amylose.

The composition may further contain resin components other than biodegradable resins, curable oligomer, additives such as several kinds of stabilizers, flame retardants, antistatic agents, fungicides and tackifiers, glass fiber, glass beads, metal powder, inorganic or organic fillers such as talc, mica and silica, and coloring agents such as pigment and dye.

In the abovementioned step of producing the polylactic acid composition, a known method such as Banbury® Mixer, a kneading machine and an open roll kneader is used to mix the polylactic acid, the plasticizer containing a rosin derivative as its main ingredient, the cross-linking monomer and other desired ingredients in the first aspect of the present invention, and to mix the polylactic acid, the plasticizer containing a dicarboxylic acid derivative and/or a glycerin derivative, the cross-linking monomer and other desired ingredients in the second aspect of the present invention.

More specifically, polylactic acid is first heated to temperatures equal to or higher than its melting point until it is softened, and then, in the first aspect of the present invention, the plasticizer containing a rosin derivative as its main ingredient, the cross-linking monomer and other desired ingredients are added thereto, whereas in the second aspect of the present invention, the plasticizer containing a dicarboxylic acid derivative and/or a glycerin derivative, the cross-linking monomer and other desired ingredients are added thereto, and subsequently the obtained mixture is kneaded.

Any kneading time may be employed depending on the kinds of the plasticizer and the cross-linking monomer and the kneading temperature. Also, the order of mixing these ingredients is not particularly limited, so that it is acceptable that all the ingredients are mixed with each other at one time and that some of the ingredients are first mixed with each other and then the remaining ingredients are added to the resulting mixture.

As the next step, in both first and second aspects of the present invention, the polylactic acid composition obtained in the previous step is molded into a molded product having a desired shape.

The method of molding is not particularly limited, allowing any appropriate known method. For example, a known molding machine such as an extruder, a compression molding machine, a vacuum forming machine, a blow molding machine, a flat die extruder, an injection molding machine and an inflation molding machine may be used.

Subsequently, in both first and second aspects of the present invention, the obtained molded product is exposed to ionizing radiation. This irradiation of ionizing radiation couples polylactic acid chains with each other, and in the case where a plasticizer containing a glycerin derivative is used as the plasticizer, also couples polylactic acid chains with the plasticizer molecules via cross-linking, thus resulting in completion of the cross-linked material of polylactic acid.

Gamma-rays, X-rays, ε-rays and α-rays may be used as the ionizing radiation, with γ-ray irradiation using Cobalt-60 and electron irradiation using an electron accelerator being preferable in industrial manufacturing.

The irradiation of ionizing radiation is conducted preferably in an air-free inert gas or under vacuum, because inactivation of activated species generated by the irradiation of ionizing radiation due to binding thereof to oxygen in the air would reduce the efficiency of cross-linking reactions.

In the first aspect of the present invention, the ionizing radiation dose is preferably in the range of 10 kGy to 100 kGy.

Depending on the content ratio of the cross-linking monomer, cross-linking of polylactic acid chains may be observed even when the ionizing radiation dose is at least 1 kGy and less than 10 kGy. However, the ionizing radiation at a dose of 10 kGy or higher would consistently cross-link a sufficient number of polylactic acid chains for maintaining the shape of the product at high temperatures equal to or higher than 60° C. At the same time, the ionizing radiation dose is preferably 100 kGy or lower because doses higher than 100 kGy promote decomposition of polylactic acid resin, which has the property of being decomposed when exposed to radiation alone, rather than the cross-linking reactions.

On the other hand, in the second aspect of the present invention, the ionizing radiation dose is preferably in the range of 10 kGy to 200 kGy.

Depending on the content ratio of the cross-linking monomer, cross-linking of polylactic acid chains may be observed even when the ionizing radiation dose is at least 1 kGy and less than 10 kGy. However, an ionizing radiation dose of 10 kGy or higher is preferable for cross-linking polylactic acid chains sufficiently enough to prevent the strength of the product from decreasing at temperatures equal to or higher than 60° C., the glass transition temperature of polylactic acid. In addition, the ionizing radiation dose of 50 kGy or higher is more preferable for cross-linking nearly 100% of polylactic acid chains. The ionizing radiation dose of 80 kGy or higher is most preferable for the complete cross-linking.

At the same time, the ionizing radiation dose is preferably 200 kGy or lower because doses higher than 200 kGy promote decomposition of polylactic acid resin, which has the property of being decomposed when exposed to radiation alone, rather than the cross-linking reactions. The ionizing radiation dose is preferably 150 kGy or lower, and more preferably 100 kGy or lower.

The third aspect of the present invention provides a cross-linked material of polylactic acid produced by the production method according to the first aspect of the present invention.

Also, the fourth aspect of the present invention provides a cross-linked material of polylactic acid produced by the production method according to the second aspect of the present invention.

In the third aspect of the present invention, the content ratio of the abovementioned plasticizer containing a rosin derivative in 100 wt % of polylactic acid is preferably in the range of 15 wt % to 30 wt %, and the abovementioned cross-linking monomer is preferably allylic-type monomer, which is contained in 100 wt % of polylactic acid at a content ratio from 3 wt % to 8 wt %.

Also, the third aspect of the present invention preferably has characteristics comparable to those of general-purpose plastics at temperatures equal to or lower than the glass transition temperature of polylactic acid. Its percentage elongation after fracture, which provides an index of these characteristics, at temperatures equal to or lower than the glass transition temperature of polylactic acid is preferably 100% or higher, more preferably 200% or higher, and most preferably 300% or higher. Furthermore, its breaking strength at temperatures equal to or lower than the glass transition temperature of polylactic acid is preferably 25 MPa or higher, and more preferably 30 MPa or higher.

Moreover, in the third aspect of the present invention, the gel fraction, which provides an index of the capability of maintaining its shape consistently even at high temperatures higher than 60° C., the glass transition temperature of polylactic acid, is 20% or higher, and preferably 30% or higher. More preferably, the gel fraction is 50% or lower because an excessively high gel fraction causes the percentage elongation after fracture to be reduced. The gel fraction is measured by the method shown in the examples described later.

On the other hand, in the fourth aspect of the present invention, the content ratio of the abovementioned plasticizer containing a dicarboxylic acid derivative and/or a glycerin derivative in 100 wt % of polylactic acid is preferably in the range of 3 wt % to 30 wt %, and the abovementioned cross-linking monomer is preferably allylic-type monomer, which is contained in 100 wt % of polylactic acid at a ratio from 3 wt % to 15 wt %.

Additionally, in the abovementioned cross-linked material of polylactic acid, the polylactic acid chains and the glycerin derivative molecules are preferably coupled with each other.

The third and fourth aspects of the present invention preferably have the characteristics of displaying no thermal absorption at the glass transition temperature of polylactic acid and exhibiting no thermal absorption associated with crystal melting at a temperature around the melting point of polylactic acid in the calorimetrical analysis performed over the temperature range from 40° C. to 200° C. using a differential scanning calorimeter.

The absence of thermal absorption at 60° C., the glass transition temperature of polylactic acid, and the absence of thermal absorption associated with crystal melting at a temperature around 160° C., the melting point of polylactic acid, described above would result in stable characteristics that vary only slightly around the glass transition temperature and the melting point of polylactic acid and enable maintaining the shape of the product even at a high temperature while ensuring the adequate flexibility at room temperature.

In addition, the cross-linked material of polylactic acids according to the third and fourth aspects of the present invention are transparent when they are prepared as described above.

Further, the fifth aspect of the present invention provides a cross-linked material of polylactic acid that may be prepared by a method other than the production method according to the first aspect of the present invention; contains polylactic acid, a plasticizer containing a rosin derivative, and cross-linking monomer; and displays a gel fraction that is at least 20% and less than 50%.

Moreover, the sixth aspect of the present invention provides a cross-linked material of polylactic acid that may be prepared by a method other than the production method according to the second aspect of the present invention; contains polylactic acid, a dicarboxylic acid derivative and/or a glycerin derivative, which are contained in 100 wt % of the polylactic acid at a content ratio from 3 wt % to 30 wt %, and cross-linking monomer, which is contained in 100 wt % of the polylactic acid at a content ratio from 3 wt % to 15 wt %; and

displays a gel fraction that is in the range of 80% to 100%.

In the sixth aspect of the present invention, the plasticizer containing a dicarboxylic acid derivative and/or a glycerin derivative as its main ingredient(s) provides the flexibility comparable to that of vinyl chloride at room temperature, and the gel fraction of 80% or higher provides the capability of maintaining the shape of the product at a high temperature of 60° C. or higher, so that the sixth aspect of the present invention can make the flexibility and the capability of maintaining the shape compatible with each other.

Also, the abovementioned cross-linked material of polylactic acid preferably has the characteristic of displaying no thermal absorption at the glass transition temperature of polylactic acid and exhibiting no thermal absorption associated with crystal melting at a temperature around the melting point of polylactic acid in the calorimetrical analysis performed over the temperature range from 40° C. to 200° C. by using a differential scanning calorimeter, thus being insensitive to temperature variations. Additionally, the fifth aspect of the present invention is transparent when it is prepared as described above.

### EFFECT OF THE INVENTION

The cross-linked material of polylactic acid prepared according to the first or second aspect of the present invention consistently maintains its shape even at high temperatures higher than 60° C., the glass transition temperature of polylactic acid, because of the cross-linking network constructed by cross-linking with irradiation of ionizing radiation. Also, it displays the flexibility comparable to that of general-purpose plastics at temperatures lower than the glass transition temperature of polylactic acid.

In particular, addition of the plasticizer containing a dicarboxylic acid derivative and/or a glycerin derivative to polylactic acid results in coupling of the polylactic acid chains and the plasticizer molecules dispersed in the cross-linking network of polylactic acid via cross-linking, thus eliminating interactions between the polylactic acid chains and providing the flexibility comparable to that of general-purpose plastics even at temperatures lower than the glass transition temperature of polylactic acid, as described above.

Meanwhile, in the sixth aspect of the present invention, the plasticizer containing a dicarboxylic acid derivative and/or a glycerin derivative as its main ingredient(s) provides the flexibility comparable to that of vinyl chloride at room temperature, and the gel fraction of 80% or higher provides the capability of maintaining the shape of the product at a high temperature of 60° C. or higher, so that the sixth aspect of the present invention can make the flexibility and the capability of maintaining the shape compatible with each other.

Consequently, the cross-linked material of polylactic acid according to the present invention can be utilized in general applications using plastics, in particular, ones using flexible polyvinyl chloride, such as rubber suction cups.

It can be used also as a shape-memory material, which requires both the flexibility and the shape-memory property.

In addition, its glass transition temperature can also be controlled by changing the ionizing radiation dose, so that the temperature at which the hardness of the product changes can be freely controlled. As a result, the present invention can be applied also to toys.

Furthermore, the biodegradability of the cross-linked material of polylactic acid of the present invention significantly reduces the adverse effects of the product on ecologies in the natural world, thus resolving the disposal issues unavoidable in known plastics. Moreover, the cross-linked material of polylactic acid of the present invention has unique characteristics of being transparent, which have not been achieved by other kinds of biodegradable resins. Also, this material has no adverse effects on living bodies, and thus can be employed for manufacturing medical devices used in and out of living bodies, such as syringes and catheters.

### BRIEF DESCRIPTION OF THE DRAWINGS

FIG. 1 is a graph showing the relationship between the ionizing radiation dose and the gel fraction as well as the relationship between the ionizing radiation dose and the percentage elongation after fracture, where the content ratio of the plasticizer containing a rosin derivative as its main ingredient is 15 wt %. The double-headed arrow indicates the range of optimum ionizing radiation doses.

FIG. 2 is a graph showing the relationship between the ionizing radiation dose and the gel fraction as well as the relationship between the ionizing radiation dose and the percentage elongation after fracture, where the content ratio of the plasticizer containing a rosin derivative as its main ingredient is 18 wt %. The double-headed arrow indicates the range of optimum ionizing radiation doses.

FIG. 3 is a graph showing the relationship between the ionizing radiation dose and the gel fraction as well as the relationship between the ionizing radiation dose and the percentage elongation after fracture, where the content ratio of the plasticizer containing a rosin derivative as its main ingredient is 20 wt %. The double-headed arrow indicates the range of optimum ionizing radiation doses.

FIG. 4 is a graph schematically showing the endothermic peak curves obtained by the analyses using a differential scanning calorimeter during the first heating phase.

FIG. 5 is a graph schematically showing the endothermic peak curves obtained by the analyses using a differential scanning calorimeter during the second heating phase.

### BEST MODE FOR CARRYING OUT THE INVENTION

A method of producing the cross-linked material of polylactic acid according to the first embodiment of the present invention is described below.

This method consists of a step of producing a polylactic acid composition by mixing polylactic acid, a plasticizer containing a rosin derivative as its main ingredient, and cross-linking monomer, and then kneading the resulting mixture;

a step of producing a polylactic acid molded product by molding the polylactic acid composition obtained in the previous step into a desired shape; and

a step of cross-linking polylactic acid chains existing in the polylactic acid molded product obtained in the previous step by irradiation of ionizing radiation.

In the step of producing the polylactic acid composition, homopolymer of lactic acid is used. When measured by differential scanning calorimetry (DSC), the melting point of polylactic acid used in the present invention is preferably 150° C. or higher, and more preferably 160° C. or higher. Additionally, the melt mass flow rate (MFR) measured at 190° C. according to the American Society for Testing and Materials (ASTM) standard D-1238 is preferably 1 g to 5 g per 10 minutes.

The abovementioned polylactic acid is softened by heating, or dissolved or dispersed in any solvent that can dissolve polylactic acid, such as chloroform and cresol.

The plasticizer containing a rosin derivative as its main ingredient is then added. The content ratio of the plasticizer in 100 wt % of polylactic acid is preferably in the range of 15 wt % to 20 wt %. The added plasticizer is uniformly dispersed by agitation and mixing.

After the addition of the plasticizer, the cross-linking monomer is added. A particularly preferable cross-linking monomer is TAIC. The content ratio of the cross-linking monomer in 100 wt % of polylactic acid is preferably in the range of 5 wt % to 8 wt %. The added cross-linking monomer is uniformly dispersed by agitation and mixing.

Subsequently, the solvent is optionally removed by drying.

It should be noted that the cross-linking monomer may be added to and mixed with the polylactic acid prior to the addition of the abovementioned derivative.

Thus the polylactic acid composition containing at least polylactic acid, a plasticizer containing a rosin derivative as its main ingredient, and cross-linking monomer is prepared.

The polylactic acid composition is softened once again by heating or other means, and then molded into a molded product having a desired shape, such as a sheet, a film, fiber, a tray, a container and a bag. This step of molding the prepared composition may be carried out, for example, with the composition being dissolved in the solvent or after cooling the composition or removing the solvent by drying. The method of molding is not particularly limited, allowing any appropriate known method. For example, a known molding machine such as an extruder, a compression molding machine, a vacuum forming machine, a blow molding machine, a flat die extruder, an injection molding machine and an inflation molding machine may be used.

As the next step, the obtained molded product is exposed to ionizing radiation to produce the cross-linked material of polylactic acid.

The ionizing radiation is preferably electron radiation generated using an electron accelerator.

The ionizing radiation dose falls within a range from 10 kGy to 100 kGy and is determined depending on the content ratio of the plasticizer containing a rosin derivative as its main ingredient and other conditions. A particularly preferable ionizing radiation dose is one that results in the cross-linked material of polylactic acid having the gel fraction that is in the range of 20% to 50% and the percentage elongation after fracture at temperatures equal to or lower than the glass transition temperature of 100% or higher.

More specifically, when the plasticizer containing a rosin derivative as its main ingredient is contained in 100 wt % of polylactic acid at a content ratio of 15 wt %, a particularly preferable dose is in the range of 5 kGy to 15 kGy. When the plasticizer containing a rosin derivative as its main ingredient is contained in 100 wt % of polylactic acid at a content ratio of 18 wt %, a particularly preferable dose is in the range of 20 kGy to 55 kGy. When the plasticizer containing a rosin derivative as its main ingredient is contained in 100 wt % of polylactic acid at a content ratio of 20 wt %, a particularly preferable dose is in the range of 50 kGy to 80 kGy.

The cross-linked material of polylactic acid obtained in the method described above has the characteristics of displaying no thermal absorption at the glass transition temperature of polylactic acid and exhibiting no thermal absorption associated with crystal melting at a temperature around the melting point of polylactic acid in the calorimetrical analysis performed over the temperature range from 40° C. to 200° C. using a differential scanning calorimeter.

Then, a method of producing the cross-linked material of polylactic acid according to the second embodiment of the present invention is described below.

This method consists of a step of producing a polylactic acid composition by mixing polylactic acid, a plasticizer containing a dicarboxylic acid derivative and/or a glycerin derivative, and cross-linking monomer, and then kneading the resulting mixture;

a step of producing a polylactic acid molded product by molding the polylactic acid composition obtained in the previous step into a desired shape; and

a step of cross-linking between polylactic acid chains in the polylactic acid molded product obtained in the previous step by irradiation of ionizing radiation.

The polylactic acid used in the step of producing the polylactic acid composition is similar to that used in the first embodiment, and it is softened by heating, or dissolved or dispersed in any solvent that can dissolve polylactic acid, such as chloroform and cresol.

The plasticizer containing a dicarboxylic acid derivative and/or a glycerin derivative is then added. The content ratio of the plasticizer in 100 wt % of polylactic acid is preferably in the range of 3 wt % to 30 wt %. The added plasticizer is uniformly dispersed by agitation and mixing.

After the addition of the plasticizer, the cross-linking monomer similar to that used in the first embodiment (TAIC) is added. The content ratio of the cross-linking monomer in 100 wt % of polylactic acid is preferably in the range of 5 wt % to 15 wt %. The added cross-linking monomer is uniformly dispersed by agitation and mixing.

Thus the polylactic acid composition containing at least polylactic acid, a plasticizer containing a dicarboxylic acid derivative and/or a glycerin derivative, and cross-linking monomer is prepared.

In a way similar to that in the first embodiment, the polylactic acid composition is molded into a molded product having a desired shape, and then the obtained molded product is exposed to ionizing radiation to produce the cross-linked material of polylactic acid.

The ionizing radiation dose falls within a range from 10 kGy to 200 kGy and is determined depending on the content ratio of the cross-linking monomer and other conditions. A particularly preferable ionizing radiation dose is one that results in the cross-linked material of polylactic acid having the gel fraction of substantially 100%.

This cross-linking process couples the polylactic acid chains with each other and also couples the glycerin derivative molecules contained in the plasticizer with the polylactic acid chains via cross-linking.

As described above, the cross-linked material of polylactic acid according to the present invention preferably shows that almost all of the polylactic acid chains and the glycerin derivative molecules contained therein are coupled with each other via cross-linking.

In other words, when the cross-linked material of polylactic acid contains the polylactic acid, the glycerin derivative and the cross-linking monomer only, its gel fraction is preferably substantially 100%.

On the other hand, when some ingredients other than the polylactic acid, the glycerin derivative and the cross-linking monomer are contained in the cross-linked material of polylactic acid, the ingredients are evaluated for the solubility in chloroform, which is a solvent used in the measurement of the gel fraction, and then the gel fraction obtained for the biodegradable cross-linked material is corrected in accordance with the following equation. The corrected gel fraction, which indicates the degree of cross-linking between the polylactic acid chains and the glycerin derivative molecules, is preferably substantially 100%.

Corrected gel fraction (%)={(Dry mass of the gel−α)/(Dry mass of the cross-linked material of polylactic acid−α−β)}×100
where α is the total mass of ingredients insoluble or hardly soluble in chloroform other than polylactic acid, a glycerin derivative and cross-linking monomer; and β is the total mass of ingredients soluble in chloroform other than polylactic acid, a glycerin derivative and cross-linking monomer.

The cross-linked material of polylactic acid obtained in the method described above has the characteristics of displaying no thermal absorption at the glass transition temperature of polylactic acid and exhibiting no thermal absorption associated with crystal melting at a temperature around the melting point of polylactic acid in the calorimetrical analysis performed over the temperature range from 40° C. to 200° C. using a differential scanning calorimeter.

The present invention is described in detail below with reference to the examples and the comparative examples. However, the present invention is not limited to these examples.

### EXAMPLE 1

The pellet-like polylactic acid, LACEA H-280, manufactured by Mitsui Chemicals was used as the polylactic acid. The plasticizer containing a rosin derivative as its main ingredient (“Lactcizer GP-2001” manufactured by Arakawa Chemical industries, LTD.) and TAIC, a kind of allylic-type cross-linking monomer, were prepared and then added to the polylactic acid by melt-extruding the mixture of the polylactic acid and the plasticizer using an extruder (PCM30 manufactured by Ikegai LTD.) at the cylinder temperature of 160° C. while titrating the TAIC at a constant rate to the pellet supply portion of the extruder using a perista pump.

The content ratios of the plasticizer containing a rosin derivative as its ingredient and the TAIC in 100 wt % of polylactic acid were respectively adjusted to 15 wt % and 7 wt %. The extruded product was cooled in water and then palletized using a pelletizer to produce a pellet-like polylactic acid composition containing polylactic acid, a plasticizer and cross-linking monomer.

This polylactic acid composition was heat-pressed into a sheet at 160° C. and then rapidly cooled in water to obtain a sheet having a thickness of 500 μm.

This sheet was exposed to electron radiation of 10 kGy in an air-free inert gas using an electron accelerator (accelerating voltage 10 MeV, current 12 mA) to complete the cross-linked material of polylactic acid according to the present invention.

### EXAMPLE 2

The cross-linked material of polylactic acid was obtained in the same way as that used in Example 1 except for that the content ratio of the plasticizer containing a rosin derivative as its main ingredient was 18 wt % relative to 100 wt % of the polylactic acid and the electron radiation dose was 30 kGy.

### EXAMPLE 3

The same procedures as those used in Example 1 were employed except for that the content ratio of the plasticizer containing a rosin derivative as its main ingredient was 20 wt % relative to 100 wt % of the polylactic acid and the electron radiation dose was 60 kGy.

### COMPARATIVE EXAMPLES 1 TO 3

The same procedures as those used in Examples 1 to 3 were employed except for that the plasticizer containing a lactic acid derivative as its main ingredient (“Lactcizer GP-4001” manufactured by Arakawa Chemical industries, LTD.) was used as the plasticizer.

### COMPARATIVE EXAMPLE 4

The same procedures as those used in Example 1 were employed except for that the electron radiation dose was 0 kGy (the electron radiation was not irradiated).

The cross-linked material of polylactic acids obtained in Examples 1 to 3 and Comparative Examples 1 to 4 were evaluated for the gel fraction and the tensile strength according to the following methods.

### [Gel Fraction Measurement]

The dry mass of each of the cross-linked material of polylactic acids was accurately measured, and then each cross-linking agent was wrapped in a 200-mesh stainless steel mesh, boiled in chloroform for 48 hours to obtain the gel separated from the sol dissolved in the chloroform. Each gel was dried at 50° C. for 24 hours to remove chloroform remaining in the gel, and then the dry mass of the gel was measured. Based on the measured dry mass, the gel fraction was calculated in accordance with the following equation.

Gel fraction (%)=(Dry mass of the gel/Dry mass of the cross-linked material of polylactic acid)×100

[Tensile Test]

Each sheet was cut into a rectangular sample measuring 1 cm in width and 10 cm in length, and the obtained sample was evaluated for the breaking strength and the percentage elongation after fracture by applying a tensile force to the sample with the gauge length 2 cm and the tensile speed 10 mm per minute.

This test was performed at 25° C.

Breaking strength (MPa)=Tensile strength at fracture (kgf)×Width of the sample (cm)×Thickness of the sample (cm)×0.098

Percentage elongation after fracture (%)={(Gauge length at fracture (cm)−2)/2}×100

The results of the measurements described above are summarized in Table I below.

TABLE I

| | | | | | | Tensile test | |
|---|---|---|---|---|---|---|---|
| | | Plasticizer | | Electron | | | Percentage |
| | | | Content | radiation | Gel | Breaking | elongation |
| | | Main | ratio | dose | fraction | strength | after fracture |
| | | ingredient | (wt %) | (kGy) | (%) | (MPa) | (%) |
| Example | 1 | Rosin | 15 | 10 | 32 | 38 | 330 |
| | 2 | derivative | 18 | 30 | 33 | 35 | 322 |
| | 3 | | 20 | 60 | 34 | 30 | 303 |
| Comparative | 1 | Lactic acid | 15 | 10 | 0.6 | 24 | 490 |
| Example | 2 | derivative | 18 | 30 | 0.4 | 10 | 530 |
| | 3 | | 20 | 60 | 0.7 | 6 | 580 |
| | 4 | Rosin | 15 | 0 | 0.4 | 36 | 362 |
| | | derivative | | | | | |

The cross-linked material of polylactic acids prepared in Examples 1 to 3 displayed the breaking strengths at least 30 MPa and the percentage elongations after fracture higher than 300%, thus achieving the characteristics comparable to those of general-purpose plastics.

Based on the measured gel fractions, which ranged from 32% to 34%, the abovementioned cross-linking agents also has the characteristics of maintaining their shape even at high temperatures equal to or higher than 60° C.

On the other hand, the sheets obtained in Comparative Examples 1 to 3, where the plasticizer containing a lactic acid derivative as its main ingredient was used, and the sheet obtained in Comparative Example 4, where the content ratio of the plasticizer was the same as that in Example 1 but the irradiation of ionizing radiation was omitted, showed the extremely low gel fractions in spite of the large percentage elongations after fracture. This suggests that little or no cross-linking reactions occurred in these comparative examples. Accordingly, these sheets may have difficulties in maintaining their shape at temperatures equal to or higher than the glass transition temperature of polylactic acid, more specifically, at high temperatures equal to or higher than 60° C.

### EXAMPLE 4

As seen in the comparison between Example 1 and Comparative Example 4, the gel fraction and the percentage elongation after fracture may vary depending on the electron radiation dose even if the same kind and content of the plasticizer is used. In the light of the findings, the following experiment was carried out to clarify the relationship between the electron radiation dose and the gel fraction, and the relationship between the electron radiation dose and the percent elongation after fracture.

The sheets having a thickness of 500 μm were produced in the same way as that used in Example 1 and these sheets were then exposed to electron radiation in a way similar to that used in Example 1 while changing the electron radiation dose in eight steps, i.e., using the doses of 0 kGy (the electron radiation was not irradiated), 10 kGy, 30 kGy, 60 kGy, 90 kGy, 120 kGy, 150 kGy and 200 kGy to produce eight types of the cross-linked material of polylactic acids.

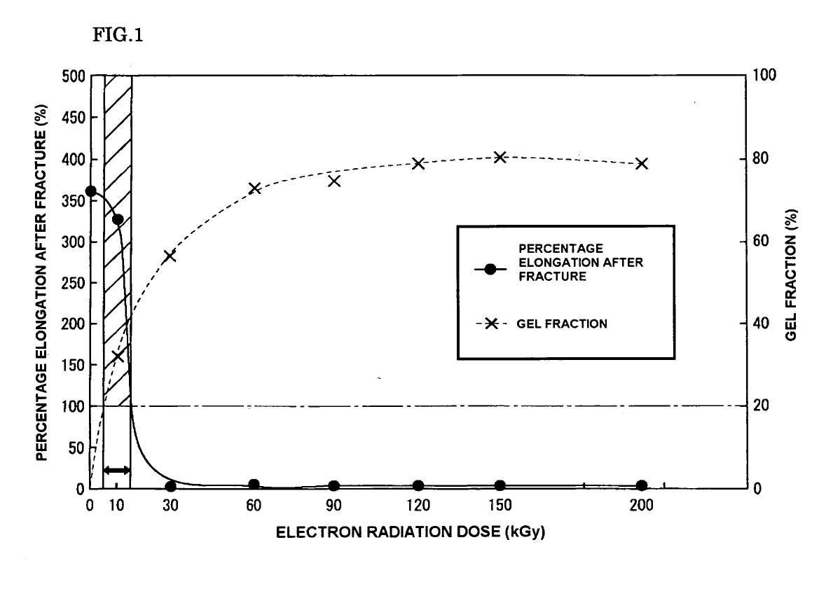

The eight types of the cross-linked material of polylactic acids were evaluated for the gel fraction and the percentage elongation after fraction according to the methods described above. FIG. 1 is a graph showing the relationship between the electron radiation dose and the gel fraction, and the relationship between the electron radiation dose and the percent elongation after fracture. The shaded area in FIG. 1 represents the range of the optimum electron radiation doses for the cross-linked material of polylactic acid of the present invention.

### EXAMPLE 5

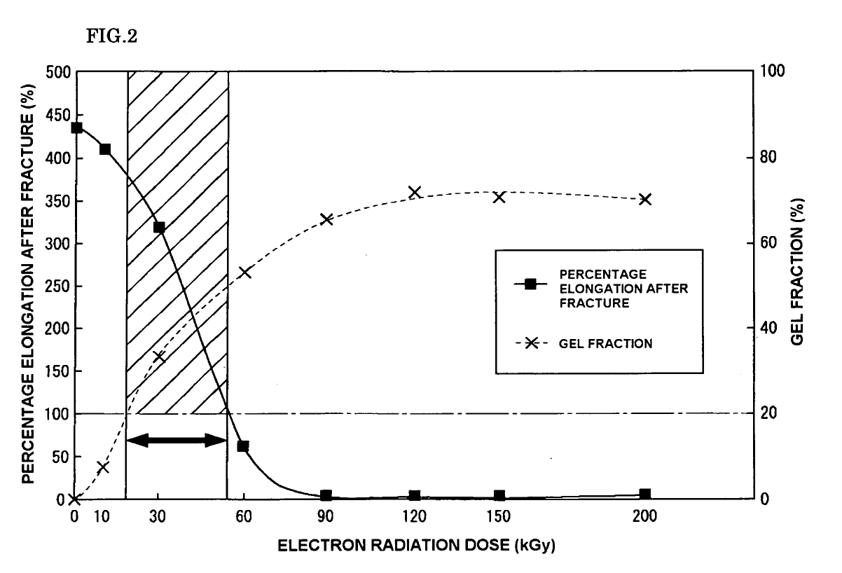

The same experiment as that in Example 4 was carried out except for that the content ratio of the plasticizer containing a rosin derivative as its main ingredient was 18 wt %, the content ratio used in Example 2. FIG. 2 is a graph showing the relationship between the electron radiation dose and the gel fraction, and the relationship between the electron radiation dose and the percent elongation after fracture. The shaded area in FIG. 2 represents the range of the optimum electron radiation doses for the cross-linked material of polylactic acid of the present invention.

### EXAMPLE 6

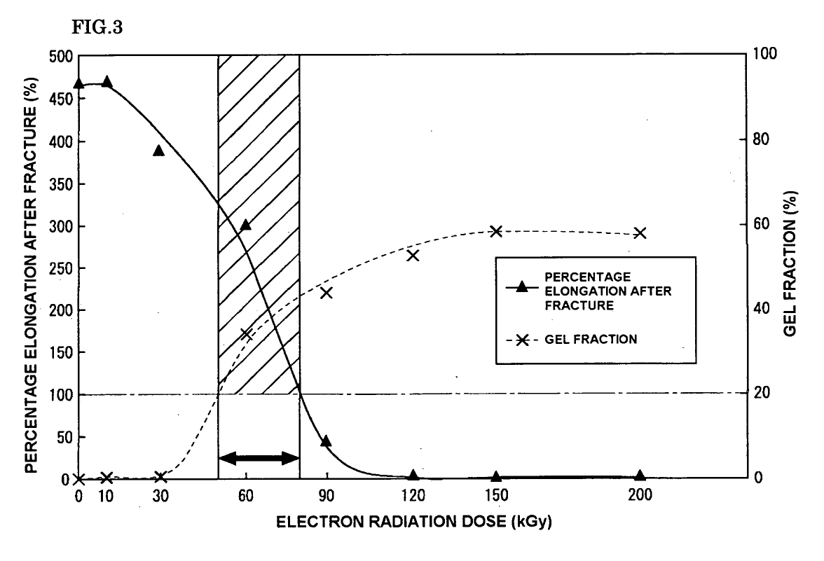

The same experiment as that in Example 4 was carried out except for that the content ratio of the plasticizer containing a rosin derivative as its main ingredient was 20 wt %, the content ratio used in Example 3. FIG. 3 is a graph showing the relationship between the electron radiation dose and the gel fraction, and the relationship between the electron radiation dose and the percent elongation after fracture. The shaded area in FIG. 3 represents the range of the optimum electron radiation doses for the cross-linked material of polylactic acid of the present invention.

The graphs shown in FIGS. 1 to 3 indicate that there are some optimum electron radiation doses for a given content of the plasticizer when it is assumed that the percentage elongation after fracture required for general-purpose plastics is 100% or higher and the gel fraction at which substantially effective cross-linking is achieved for maintaining the shape of the product even at high temperatures equal to or higher than 60° C. is in the range of 20% to 50%.

More specifically, when the content ratio of the plasticizer containing a rosin derivative as its main ingredient is 15 wt %, a preferable electron radiation dose is in the range of 5 kGy to 15 kGy.

When the content ratio of the plasticizer containing a rosin derivative as its main ingredient is 18 wt %, a preferable electron radiation dose is in the range of 20 kGy to 55 kGy.

When the content ratio of the plasticizer containing a rosin derivative as its main ingredient is 20 wt %, a preferable electron radiation dose is in the range of 50 kGy to 80 kGy.

### EXAMPLE 7

The sheets having a thickness of 500 μm were produced in the same way as in Example 3 and these sheets were then exposed to electron radiation in a way similar to that in Example 1 while changing the electron radiation dose in eight steps, i.e., using the doses of (1) 0 kGy (the electron radiation was not irradiated), (2) 5 kGy, (3) 10 kGy, (4) 20 kGy, (5) 30 kGy, (6) 60 kGy, (7) 90 kGy and (8) 120 kGy to produce eight types of the cross-linked material of polylactic acids.

The eight types of the cross-linked material of polylactic acids (1) to (8) were subjected to the measurement of the endothermic peak using a differential scanning calorimeter while increasing the temperature from 0° C. to 100° C.

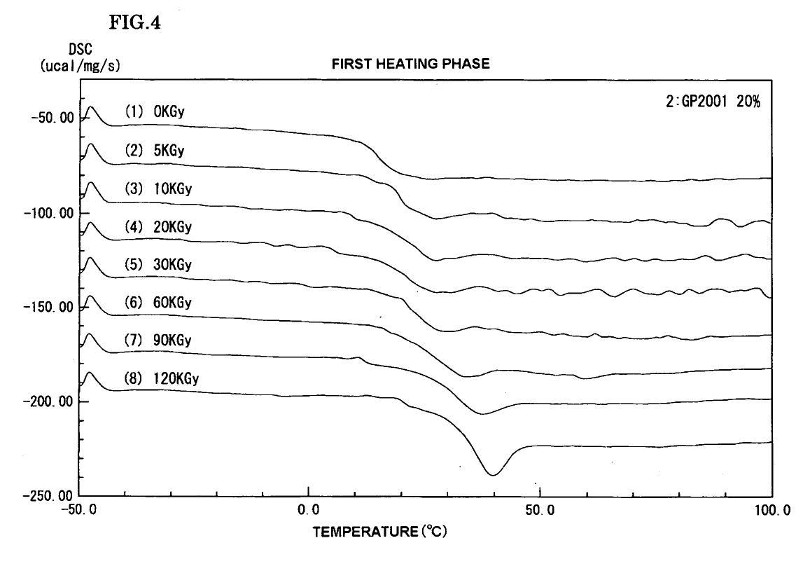

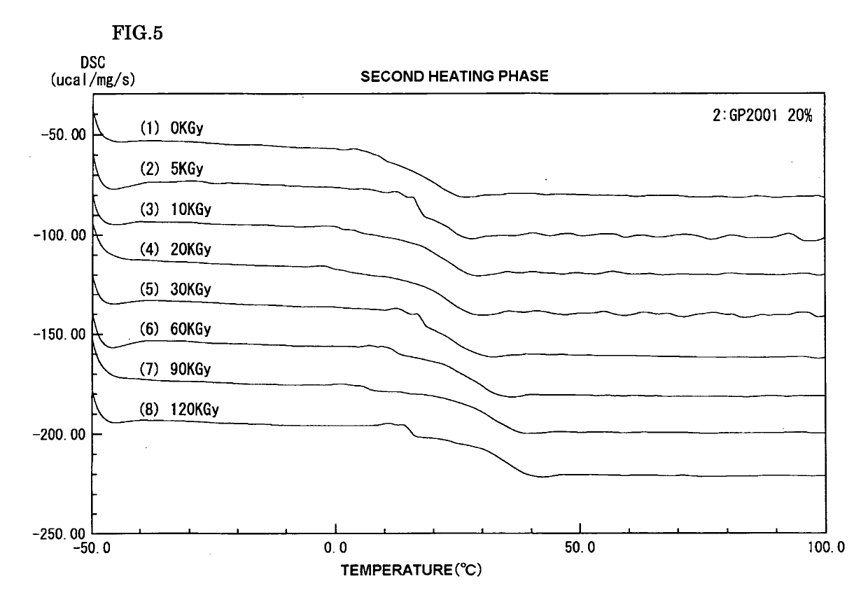

FIGS. 4 and 5 show the results. FIG. 4 schematically shows the endothermic peak curves obtained during the first heating phase, while FIG. 5 schematically shows the endothermic peak curves obtained during the second heating phase.

As seen in FIGS. 4 and 5, adding the plasticizer to the polylactic acid resulted in lowering of the glass transition temperature peaks from around 60° C. to around the room temperature, 25° C. However, the irradiation of electron radiation caused the peaks to rise with the electron radiation dose.

### EXAMPLE 8

Similarly to Example 1, the pellet-like polylactic acid, LACEA H-400, manufactured by Mitsui Chemicals was used as the polylactic acid. The plasticizer containing a dicarboxylic acid derivative as its main ingredient (“DAIFFATY-101” manufactured by Daihachi Chemical Industry Co., Ltd.) and TAIC, a kind of allylic-type cross-linking monomer, were prepared and then added to the polylactic acid by melt-extruding the mixture of the polylactic acid and the plasticizer using an extruder (PCM30 manufactured by Ikegai LTD.) at the cylinder temperature of 160° C. while titrating the TAIC at a constant rate to the pellet supply portion of the extruder using a perista pump.

The content ratios of the plasticizer containing a dicarboxylic acid derivative as its ingredient and the TAIC in 100 wt % of polylactic acid were respectively adjusted to 10 wt % and 7 wt %. The extruded product was cooled in water and then palletized using a pelletizer to produce a pellet-like polylactic acid composition containing polylactic acid, a plasticizer and cross-linking monomer.

In a similar way to that used in Example 1, this composition was heat-pressed into a sheet at 160° C. and then rapidly cooled in water to obtain a sheet having a thickness of 500 μm.

This sheet was exposed to electron radiation of 100 kGy in an air-free inert gas using an electron accelerator (accelerating voltage 10 MeV, current 12 mA) to complete the cross-linked material of polylactic acid according to the present invention.

### EXAMPLE 9

The cross-linked material of polylactic acid was obtained in the same way as that used in Example 8 except for that the content ratio of the plasticizer containing a dicarboxylic acid derivative as its main ingredient was 20 wt % relative to 100 wt % of the polylactic acid. EXAMPLE 10

The same procedures as those used in Example 8 were employed except for that triacetyl glyceride (“triacetin” manufactured by Daihachi Chemical Industry Co., Ltd.), a plasticizer containing a glycerin as its main ingredient, was used as the plasticizer.

### EXAMPLE 11

The same procedures as those used in Example 9 were employed except for that triacetyl glyceride (“triacetin” manufactured by Daihachi Chemical Industry Co., Ltd.), a plasticizer containing a glycerin as its main ingredient, was used as the plasticizer.

### COMPARATIVE EXAMPLES 5 TO 8

The same procedures as those used in Examples 8 to 11 were employed except for that the irradiation of electron radiation was omitted, i.e., the electron radiation dose was 0 kGy.

### COMPARATIVE EXAMPLES 9 AND 10

The same procedures as those used in Examples 8 and 9 were employed except for that the plasticizer containing a lactic acid derivative as its main ingredient (“GP-4001” manufactured by Arakawa Chemical industries, LTD.) was used as the plasticizer.

### COMPARATIVE EXAMPLE 11

The same procedures as those used in Examples 8 were employed except for that no plasticizer was used.

Examples 8 to 11 and Comparative Examples 5 to 11 were evaluated for the gel fraction according to the methods described earlier, and evaluated for the flexibility and the heat resistance respectively in the following 90-degree bend test and the warm water immersion test.

### [90-Degree Bend Test]

Each of the sheets was cut into a stick sample measuring 1 cm in width and 15 cm in length. Each sample was held at its ends with hands and bent at 90°, maintained for a few seconds and then released. After that, the sample was evaluated for any fracture, crease and tendency to bend.

[Warm Water Immersion Test]

Each of the sheets was cut into a stick sample measuring 1 cm in width and 5 cm in length. Each sample was immersed in water at 90° C. for 5 minutes while being evaluated for any deformation.

Table II shows the results obtained in the tests described above and the production conditions used.

TABLE II

| | | Plasticizer | | Electron | | | Warm |
|---|---|---|---|---|---|---|---|
| | | | Content | radiation | Gel | | water |
| | | | ratio | dose | fraction | 90-degree | immersion |
| | | Type | (wt %) | (kGy) | (%) | bend test | test |
| Example | 8 | DAIFFATY- | 10 | 100 | 91 | Returned | Unchanged |
| | 9 | 101 | 20 | | 84 | to the | |
| | 10 | Triacetyl | 10 | | 99< | original | |
| | 11 | glyceride | 20 | | | shape | |
| Comparative | 5 | DAIFFATY- | 10 | 0 | 1> | from the | Deformed |
| Example | 6 | 101 | 20 | | 1> | bend | |
| | 7 | Triacetyl | 10 | | 1> | state | |
| | 8 | glyceride | 20 | | 1> | | |
| | 9 | GP-4001 | 10 | 100 | 1> | | |
| | 10 | | 20 | | 1> | | |
| | 11 | None | — | | 99< | Fractured | Unchanged |

In Examples 8 and 9, where the plasticizer containing a dicarboxylic acid derivative as its main ingredient was used, the gel fractions lowered by the content ratio of the plasticizer were obtained. In Examples 10 and 11, where the plasticizer containing a glycerin derivative as its main ingredient was used, the gel fractions were close to 100%, thus indicating that the plasticizer molecules and the polylactic acid chains were coupled with each other via cross-linking.

On the other hand, in Comparative Examples 5 to 8, where the irradiation of electron radiation was omitted, the gel fractions were lower than 1%, the detection limit, thus indicating that no cross-linking reactions occurred. Also in Comparative Examples 9 and 10, the gel fractions were lower than 1%, the detection limit, in spite of the irradiation of electron radiation at a dose of 100 kGy, indicating that no cross-linking reactions occurred. In Comparative Example 11, where no plasticizer was used, the gel fraction was close to 100%.

It is thus concluded that Examples 8 to 11 displaying the gel fractions of 84% or higher can maintain their shape at high temperatures equal to or higher than the glass transition temperature, whereas Comparative Examples 5 to 10 can not.

This is apparent also from the facts that, in the warm water immersion test for evaluating the samples' capability of maintaining their shape at temperatures equal to or higher than the glass transition temperature of polylactic acid, all of the examples tested could maintain their shape but Comparative Examples 5 to 10 were deformed, whereas Comparative Example 11, which contained no plasticizer, was not deformed.

Meanwhile, in the 90-degree bend test for evaluating the flexibility at temperatures lower than the glass transition temperature of polylactic acid, all of the examples and comparative examples tested except for Comparative Example 11, which contained no plasticizer, could be bent without any unrecoverable deformation.

As is obvious in the test results described above, Examples 8 to 11 have the flexibility at room temperature, which is lower than the glass transition temperature of polylactic acid, and can maintain their shape at high temperatures equal to or higher than the glass transition temperature, so that they can make the flexibility and the capability of maintaining the shape compatible with each other.

1. A method of producing a cross-linked material of polylactic acid comprising
a step of producing a polylactic acid composition by mixing polylactic acid with at least a plasticizer containing a rosin derivative and cross-linking monomer, and then kneading the resulting mixture;
a step of producing a polylactic acid molded product by molding the polylactic acid composition obtained in the previous step into a desired shape; and
a step of cross-linking the polylactic acid molded product obtained in the previous step by irradiation of ionizing radiation.

2. The method of producing a cross-linked material of polylactic acid according to claim 1, wherein the dose of the ionizing radiation is in the range of 10 kGy to 100 kGy.

3. A method of producing a cross-linked material of polylactic acid comprising
a step of producing a polylactic acid composition by mixing polylactic acid with a plasticizer containing a dicarboxylic acid derivative and/or a glycerin derivative and cross-linking monomer, and then kneading the resulting mixture;
a step of producing a polylactic acid molded product by molding the polylactic acid composition obtained in the previous step into a desired shape; and
a step of cross-linking the polylactic acid molded product obtained in the previous step by irradiation of ionizing radiation.

4. The method of producing a cross-linked material of polylactic acid according to claim 3, wherein the dose of the ionizing radiation is in the range of 10 kGy to 200 kGy.

5. A cross-linked material of polylactic acid produced using the method according to claim 1.

6. The cross-linked material of polylactic acid according to claim 5, wherein the plasticizer containing a rosin derivative is contained in 100 wt % of the polylactic acid at a content ratio from 15 wt % to 30 wt %.

7. The cross-linked material of polylactic acid according to claim 5, wherein allylic-type monomer is contained as the cross-linking monomer in 100 wt % of the polylactic acid at a content ratio from 3 wt % to 8 wt %.

8. The cross-linked material of polylactic acid according to claim 5, wherein the percentage elongation after fraction is 100% or higher at temperatures equal to or lower than the glass transition temperature of the polylactic acid.

9. The cross-linked material of polylactic acid according to claim 5, which shows no thermal absorption at the glass transition temperature of the polylactic acid and no thermal absorption associated with crystal melting at a temperature around the melting point of the polylactic acid in a calorimetrical analysis performed over the temperature range from 40° C. to 200° C. using a differential scanning calorimeter.

10. A cross-linked material of polylactic acid comprising polylactic acid, a plasticizer containing a rosin derivative, and cross-linking monomer, wherein the gel fraction thereof is at least 20% and less than 50%.

11. A cross-linked material of polylactic acid produced using the method according to claim 3.

12. The cross-linked material of polylactic acid according to claim 11, wherein the polylactic acid and the glycerin derivative are coupled with each other via cross-linking.

13. The cross-linked material of polylactic acid according to claim 11, wherein the plasticizer containing a dicarboxylic acid derivative and/or a glycerin derivative is contained in 100 wt % of the polylactic acid at a content ratio from 3 wt % to 30 wt %.

14. The cross-linked material of polylactic acid according to claim 11, wherein allylic-type monomer is contained as the cross-linking monomer in 100 wt % of the polylactic acid at a content ratio from 3 wt % to 15 wt %.

15. The cross-linked material of polylactic acid according to claim 11, which shows no thermal absorption at the glass transition temperature of the polylactic acid and no thermal absorption associated with crystal melting at a temperature around the melting point of the polylactic acid in a calorimetrical analysis performed over the temperature range from 40° C. to 200° C. using a differential scanning calorimeter.

16. A cross-linked material of polylactic acid comprising polylactic acid, a dicarboxylic acid derivative and/or a glycerin derivative, and cross-linking monomer, wherein the content ratio(s) of the dicarboxylic acid derivative and/or the glycerin derivative are/is in the range of 3 wt % to 30 wt % relative to 100 wt % of the polylactic acid and the content ratio of the cross-linking monomer is in the range of 3 wt % to 15 wt % relative to 100 wt % of the polylactic acid; and
the gel fraction thereof is in the range of 80% to 100%.
